(19) United States
(12) Patent Application Publication
(10) Pub. No.: US 2014/0377241 A1
(43) Pub. Date: Dec. 25, 2014

(54) BIOPREPARATION BALIS FOR THE PROPHYLAXIS AND TREATMENT OF INFECTIOUS DISEASES

(71) Applicants: Mikhail Viktorovich Dalin, Moscow (RU); Elena Aleksandrovna Vasilieva, Moscow (RU); Irina Viktorovna Anokhina, Lubertsy Moskovskaya obl. (RU); Eduard Georgievich Kravtsov, Moscow (RU); Natalia Vyacheslavovna Yashina, Moscow (RU); Kirill Mikhailovich Mefyod, Moscow (RU); Marina Aleksandrovna Kulchitskaya, Nizhny Novgorod (RU); Aleksandr Aleksandrovich Matveev, Moscow (RU); Klavdiya Nikolaevna Slinina, Nizhny Novgorod (RU); Aleksandr Sergeevich Vasiliev, Moscow (RU)

(72) Inventors: Mikhail Viktorovich Dalin, Moscow (RU); Elena Aleksandrovna Vasilieva, Moscow (RU); Irina Viktorovna Anokhina, Lubertsy Moskovskaya obl. (RU); Eduard Georgievich Kravtsov, Moscow (RU); Natalia Vyacheslavovna Yashina, Moscow (RU); Kirill Mikhailovich Mefyod, Moscow (RU); Marina Aleksandrovna Kulchitskaya, Nizhny Novgorod (RU); Aleksandr Aleksandrovich Matveev, Moscow (RU); Klavdiya Nikolaevna Slinina, Nizhny Novgorod (RU); Aleksandr Sergeevich Vasiliev, Moscow (RU); Alla Leonovna Lazovskaya, (US); Zoya Glebovna Vorobyova, (US)

(21) Appl. No.: 13/993,030

(22) PCT Filed: Feb. 7, 2012
(86) PCT No.: PCT/RU2012/000068

§ 371 Date: Jan. 10, 2014

(30) Foreign Application Priority Data
Dec. 8, 2010 (RU) 2010150270

(51) Int. Cl. A61K 35/74

(52) U.S. Cl. CPC A61K 35/74; A61K 2035/11
USPC 424/93.462; 424/93.46

(57) ABSTRACT

Biotechnology and the creation of novel probiotic preparations based on the Bacillus-type bacteria, which can be used for the prevention and treatment of infectious diseases in humans and of dysbiosis of various etiologies. The biological preparation for the prevention and treatment of infectious diseases, primarily tuberculosis, dysbiosis of various etiologies, and candidiasis, comprising live microbial mass of Bacillus subtilis VKPM B-8611 [symbol] Bacillus licheniformis VKPM B-8610 strains, wherein said preparation additionally comprises live culture filtrates of said Bacillus subtilis Bacillus licheniformis strains and a lectin-binding substance comprising a live microbial mass culture fluid supernatant of the Lactobacillus fermentum 90 TS-4(21) strain in the following component ratio: a live microbial mass of the Bacillus subtilis VKPM B-8611 strain—at least 5×10⁸ CFU; a live microbial mass of the Bacillus licheniformis VKPM B-8610 strain—at least 2×10⁸ CFU; a lyophilized filtrate of live microbial mass of the Bacillus subtilis strain VKPM B-8611, 2.5-4.5 mg; a lyophilized filtrate of live microbial mass of the Bacillus licheniformis strain VKPM B-8610, 2.5-4.5 mg; and a lyophilized lectin-binding substance including a culture fluid supernatant of live microbial mass of the Lactobacillus fermentum 90 TS-4(21) strain with the molecular weight exceeding 20 kDa. 5.5-7.5 mg, and 1 i.c., 2 d.c., 1 il. 7 tabl.

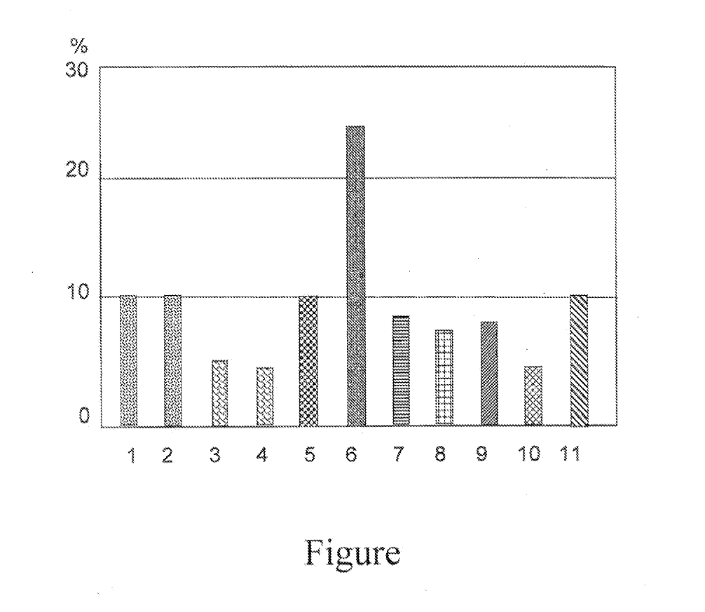

### BACKGROUND OF THE INVENTION

1. Field of the Invention

This invention relates to biotechnology and the creation of novel probiotic preparations based on the Bacillus-type bacteria, which can be used for the prevention and treatment of infectious diseases in humans and of dysbiosis of various etiologies.

2. Discussion of Related Art

Multiple drug resistance of mycobacteria to antibiotics is known to present a big problem when treating tuberculosis, which is an insurmountable obstacle to the reduction of morbidity from tuberculosis with wide drug resistance (so called XDR-TB). For XDR-TB, in addition to drug resistance characterized by multiple drug resistance, there is also resistance to all fluoroquinolones and to at least one of the three injectable second-line drugs: capreomycin, kanamycin, and amikacin (WHO Report No WHO/HTM/TB/2010.3). Presently, there are known tuberculosis mycobacteria strains resistant to three, four, five or even six antibiotics, including reserve antibiotics, making the search for novel preparations addressing this problem very urgent.

A preventive and curative probiotic preparation (Russian Patent Reference RU2314819 C1, Lyalick et al, Jan. 20, 2008) based on the Bacillus genus bacterial spore biomass and containing Bacillus subtilis RNCIM B-7048 bacterial strain. Bacillus subtilis RNCIM B-7092 bacterial strain, and/or, Bacillus licheniformis RNCIM B-7038 bacterial strain, has been described. This preparation expands the range and increases the biological activity of the preventive and curative probiotic preparation, however, it does not restore a positive response to antibiotics.

There is a known preparation (Russian Patent Reference RU2403260 C2, SPODSBERG et al., Nov. 10, 2010) (PCT Publication WO 2006/097110, Sep. 21, 2006) comprising Bacillus licheniformis as a producer with a chemical structure of a peptide. This preparation exhibits extensive antimicrobial activity against infectious agents, both prokaryotes and eukaryotes. However, this preparation has not been reported to be effective against drug-resistant tuberculosis mycobacteria strains when administered concomitantly with antibiotics.

### SUMMARY OF THE INVENTION

One objective of this invention is to provide a preparation for the treatment of infection diseases, primarily chronic tuberculosis XDR TB caused by mycobacteria with extensive drug resistance.

A biological preparation for the prevention and treatment of infectious diseases, mostly tuberculosis and concomitant candidiasis as well as dysbiosis of various etiologies, comprising live microbial mass of Bacillus subtilis VKPM B-8611 and Bacillus licheniformis VKPM B-8610 strains, wherein said preparation additionally comprises filtrates of live cultures of said Bacillus subtilis and Bacillus licheniformis strains and a lectin-binding substance comprising a culture fluid supernatant of live microbial mass of the Lactobacillus fermentum 90 TS-4(21) stain in the following component ratio:

live microbial mass of the Bacillus subtilis VKPM B-8611 strain—at least 5×10⁸ CFU;

live microbial mass of the Bacillus licheniformis VKPM 3-8610 strain—at least 1×10⁷ CFU;

lyophilized filtrate of live microbial mass of the Bacillus subtilis VKPM B-8611 strain, 2.5-4.5 mg;

lyophilized filtrate of live microbial mass of the Bacillus licheniformis VKPM B-8610 strain, 2.5-4.5 mg;

lyophilized lectin-binding substance comprising a culture fluid supernatant of live microbial mass of the Lactobacillus fermentum 90 TS-4(21) strain with the molecular weight exceeding 20 kDa, 5.5-7.5 mg.

The infectious diseases comprise chronic tuberculosis; intestinal infections, including hemorrhaging colitis caused by E. coli O-157; nosocomial surgical infections; candidiasis; and dysbiosis. Chronic tuberculosis may be caused by XDR TB mycobacteria with extensive drug resistance.

One technical result of this invention is to provide increased effectiveness of antibacterial therapy against tuberculosis infection, including XDR TB, mediated by multidrug resistant tuberculosis mycobacteria.

Bacillus subtilis VKPM B-8611 and Bacillus licheniformis VKPM B-8610 strains included in the preparation are deposited in the Federal Institution State Research and Development Institute of Genetics; the date of the international deposit is Aug. 12, 2004). Live cultures of both strains exhibit extensive antagonistic activity, high proteolytic activity, ability to produce bacteriocins, as well as lysozyme, amylase, and peptide enzymes. Bacterial filtrates of live cultures comprising the extracellular waste products exhibited an unexpected effect toward the pathogenic mycobacteria, the pathogen of tuberculosis. In addition to their antagonistic activity (ability to stunt the growth of mycobacteria), they changed the sensitivity of the pathogens to antibiotics by increasing the antibiotic activity range and by restoring sensitivity to 1, 2, 3, 4, or 5 antibiotics. Notably, one of the problems common to all tuberculosis patients undergoing long-term treatment with antibiotics is dysbiosis and candidiasis as an extreme stage of dysbiosis. The preparation of this invention is a multi-component probiotic. It additionally contains a lectin-binding complex that blocks candida adhesion to the epithelial cells of the terminal ecological niches of the macroorganism, thus preventing candidiasis. To prepare the lectin-binding substance as part of the composition of the probiotic preparation, we used a producer of the Lactobaterin probiotic preparation. We chose the Lactobacillus fermentum 90-TS-4 (21) (clone 3) variant agglutinating in the presence of Concanavalin-A and exhibiting strong ability to express the lectin-binding component into the culture medium. Precisely this glycoprotein complex prevents colonization of yeast-like fungi of Candida genus on the epithelium (see Russian Patent Reference RU2367686 C1, Anokhina et al., Sep. 20, 2009). The probiotic Balis composition restored sensitivity to antibiotics in about 80% of drug-resistant pathogenic mycobacteria, additionally, it increased sensitivity of the XDR TB strains to 1-5 antibiotics.

B. subtilis 07 (VKPM B-8611) and B. licheniformis 09 (VKPM B-8610) strains are isolated from healthy wheat plants, deposited into the Russian Collection of Industrial Microorganisms, and have the following characteristics:

Bacillus subtilis VKPM B-8611—are Gram-positive aerobic spore-forming rods, 2.7-0.6×0.8-0.7 μm in size, arranged alone or in chains. The cells are motile. In aerobic conditions, they form centrally aligned in cells oval spores. During sporulation, the cells do not become distended.

On Gauze #2 medium, wort agar, and Gromyko media the strain grows heavily and forms opaque, flesh-colored, folded colonies with jagged edges, which are easily removed from agar with a loop.

No inclusions of poly-β-hydroxybutyric acid were detected in the protoplasm of the strain in the glucose agar-grown cells. The culture forms a film on beef-extract broth.

It does not grow in anaerobic conditions, does not hydrolyze urea, does not produce gas from the nitrates in anaerobic conditions, and does not produce arginine dihydrolase. The culture forms catalase. It is Voges-Proskauer positive, grows in the presence of 7% NaCl, hydrolyzes starch and casein, fluidifies gelatin, ferments glucose, arabinose, xylose, and mannitol producing acid with no gas, reduces nitrates, decolorizes methylene blue, does not exhibit coagulase and lecithinase activity, but exhibits high proteolytic and amylase activity.

Bacillus licheniformis VKPM B-8610 are Gram-positive spore-forming rods, 2.6-0.7×0.5-0.6 μm in size. The cells are motile, peritrichous, and mostly form chains. The spores have an oval shape and align centrally in cells. During sporulation, the cells do not swell. No inclusions of poly-β-hydroxybutyric acid were detected in the protoplasm of the glucose agar-grown cells.

In beef-extract agar, it forms colonies with a dull coarse surface; they are opaque, tightly held to agar, and the surface is often slimy. It forms a film on beef-extract broth, at times with an off-white tint.

The culture forms catalase, it grows on agar in anaerobic conditions, it is Voges-Proskauer positive, grows in the presence of 7% NaCl, hydrolyzes starch and casein and does not hydrolyze urea. It slowly liquifies gelatin, ferments glucose, arabinose, xylose, and mannitol, producing acid with no gas, reduces nitrates. In anaerobic conditions, the nitrites produce gas. It produces arginine dihydrolase and lysozyme and does not exhibit coagulase and lecithinase activity.

The main properties of the B. licheniformis VKPM B-8610 and B. subtilis VKPM B-8611 strains are represented in Table 1.

B. licheniformis VKPM B-8610 and B. subtilis VKPM B-8611 strains exhibit strong antagonistic activity against a wide range of pathogenic and opportunistic microorganisms and they are resistant to many antibiotics (Table 2,3). Hence, Balis may be used concomitantly with antibiotics thus enhancing their combined antibacterial effect.

The mentioned strains can be included in the composition of Balis biological preparation in various combinations. For example, they can be used in equal ratios (according to cell titers): biomass of the B. subtilis VKPM B-8611 strain in the 1·10⁹ CFU/ml titer and biomass of the B. licheniformis strain in the 1·10⁹ CFU/ml titer, or in any ratios in the (1-100):(100-1) range, for example: biomass of the B. subtilis VKPM B-8611 strain in the 5·10⁹ CFU/ml titer and biomass of the B. licheniformis VKPM B-8610 strain in the 1·10⁹ KOE/ml titer, or biomass of the B. subtilis strain in the 1·10¹⁰ CFU/ml titer and biomass of the B. licheniformis VKPM No B-8610 strain in the 5·10⁹ CFU/ml titer, or biomass of the B. subtilis VKPM B-8611 strain in the 1·10⁹ CFU/ml titer and biomass of the B. licheniformis VKPM B-8610 strain in the 1·10⁷ CFU/ml titer, etc.

In order to obtain sterile filtrates of live cultures, B. subtilis VKPM B-8611 [symbol] B. licheniformis VKPM B-8610 strains are cultured separately with constant shaking on a reciprocating shaker at 37° C. The biomass is filtered through sterilizing filters with prior centrifuging (g=9859.6 m/sec²), pH is brought to 6.0±0.05.

The end products are extracellular waste products of the B. subtilis VKPM B-8611 and B. licheniformis VKPM B-8610 strains. The live culture filtrates are lyophilized, and the resultant substances are stored at a temperature not exceeding 20° C. The substances are stable and remain unchanged during lyophilization and also during 2 years of storage.

The lyophilized substance was used to prepare the preparation, but liquid filtrates can also be used. The substance may be included in the proposed Bolis preparation in the amount of 3 mg/ml.

In order to prepare the lectin-binding substance, the Lactobacillus fermentum 90-NS-4(21) strain was used as a producer (the strain was deposited into the Russian Collection of Industrial Microorganisms under the registration number B-7573). After the L. fermentum 90-TS-4(21) strain had been plated, the colonies agglutinating with Con A on glass in concentrations of at least 1.75·10⁻³ mg/ml were selected.

The chosen L. fermentum 90-TS-4 (21) strain variant was cultured in liquid MPC-1 medium in a 1,000 ml volume under CO₂ for 15-17 hrs. at 37 (±0.05)° C. with constant stirring.

After 15-17 hours, the cell culture was centrifuged (14-16 min.), 3,000 rpm, g=9,859.6 m/sec²) to remove the culture fluid.

The culture fluid supernatant was transferred to the AMICON (USA) cells, (2,000 ml) and separated from the low-molecular-weight components and the remaining bacterial cells with sterilizing membranes Millipore with a 0.02 μm pore diameter.

The purified culture fluid was concentrated to 100 ml in the AMICON (USA) cells (2,000 ml) by using UM-20 Diaflo membranes. At the same time, the culture fluid supernatant was washed three times with distilled water to remove lactic acid and bring the pH to 6.0±0.05.

The obtained culture fluid concentrate of the L. fermentum 90 TS-4 (21) strain was incubated for 24 hours at room temperature with Con A-sepharose. After 24 hours, the supernatant was removed and (Con A-sepharose was washed three times with phosphate buffer (pH=8.2). Then, physiological solution was added (pH=3.0) and the fluid was incubated for 3 hours at room temperature. A supernatant was collected and its pH was brought up to 7.2 with 0.1 M NaOH. The presence of lactin-binding substance was monitored with 0.1% Con A solution in the circumoval precipitin test.

The end product is an anti-adhesive component based on lectin-binding structures of the L. fermentum 90 TS-4 (21) strain with the following characteristics:

The component containing 150.0 μg/ml of protein is characterized by aggregative instability at pH over 6.0;

Molecular weight determined by polyacrylamide gel electrophoresis is 25 to 39 kDa;

The properties of the lectin-binding substance are stable and they don't change during storage and lyophilization.

The lectin-binding substance modulates adhesive activity of the C. albicans type yeast-like test-cultures and opportunistic pathogenic strains of the E. coli type microorganisms (see Russian Patent Reference RU2367686).

Modulating action of the fraction of the L. fermentum 90 TS-4 (21) strain culture fluid concentrate (CF) incapable of reacting with Con A and the fraction capable of reacting with Con A in the adhesion inhibition test of the test-culture conducted on vaginal epithelial cells (VE) revealed the following (see Table 4): The fraction of the lectin-binding substance purified by chromatography actively inhibits adhesion of the vaginal isolate of the yeast-like C. albicans 04.703 B strain type fungi on epithelial cells and less so the adhesion of the oral isolate of the yeast-like C. albicans type fungi. The fraction has no effect on the K 12 strain E. coli adhesion, but it significantly blocks the 89-1449 strain E. coli adhesion to vaginal epithelial cells. In addition, when the lectin-binding component is removed, adhesive activity of the part of the E. coli test-culture to vaginal epithelial cells is increased. Adhesion of the vaginal isolate of the yeast-like C. albicans 04.703 strain type fungi in this model significantly decreases. These data confirm that the obtained lectin-binding component exhibits a distinct modulating effect on highly adhesive strains of the yeast-like C. albicans type fungi and on other opportunistic pathogenic microorganisms.

When preparing the composition of the multi-component probiotic preparation Balis, the ratio of the VKPM B-8611 and B. licheniformis VKPM B-8610 live strains can be varied within certain limits as indicated above. Live culture filtrates of the VKPM B-8611 and B. licheniformis VKPM B-8610 strains (extracellular substances) were added in the amount of 3 mg as an average. Lectin-binding Lactobacillus fermentum 90 TS-4 (21) was added in the amount of 6 mg. Balis biological preparation additionally contains a protective medium for shielding the bacteria during the technological process, preparing the final composition and its subsequent storage. As a protective medium, the preparation may contain, for example, sucrose-gelatinous medium, dry milk, gelatose, lactose, sucrose, etc. Balis biological preparation may additionally contain a solvent. An example of a solvent used in the preparation may be distilled or boiled water, or a physiological solution, etc.

Balis preparation may additionally contain excipients commonly used in the preparation of various pharmaceutical compositions. In the tablet form, it may contain, for example, dextrans, polyglucin, starch, polyvinylpyrrolidone, sucrose, lactose, calcium stearate, glucose, sodium bicarbonate, aluminum hydroxide, methylcellulose, talc, etc. In the suppository form, it may contain, for example, the following excipients: confectionary fat, paraffin, lanolin, cocoa butter, aluminum hydroxide gel, etc.

The biological preparation may be encapsulated or immobilized on various carriers or sorbents, for example, on aerosil, cellulose, activated charcoal, carboxymethylcellulose, hydroxyethylcellulose, chitosan, etc.

The biological preparation may also be lyophilized.

Balis biological preparation may be used orally, vaginally, rectally, or topically as an aqueous suspension. The mechanism of action of Balis biological preparation is based on the adhesive and antagonistic activity of probiotic bacteria. This effect is caused by various physiologically active substances displacing pathogenic and opportunistic microorganisms from the digestive tract and stimulating specific and nonspecific defense reactions of the macroorganism.

This invention is illustrated by the following examples:

### EXAMPLE 1

### Preparing the Liquid Preparation

The strains are cultured separately or in solid or liquid growth media.

Laboratory technology: the strains are cultured on solid agar media in flasks or glass bottles in a thermostat-controlled shaker at 22° C. to 38° C. from 12-16 hrs. to up to 7 days. Upon completion of the incubation, the biomass grown on top of the growth medium is washed off with a stabilizer—a protective medium containing a 5% lactose solution, combined together in the 10:1 ratio, and poured into flasks. The obtained preparation contains biomass of the B. subtilis VKPM B-8611 strain in the 1·10¹⁰ CFU/ml titer and biomass of the B. licheniformis VKPM B-8610 strain in the 1·10⁹ CFU/ml titer. 2.5-4.5 mg/ml of each of the filtrates of the aforementioned strains and 5.5-7.5 mg of the Lactobacillus fermentum 90 TS-4 (21) lectin-binding substance are added to the obtained bacterial composition.

Industrial technology: the strains are cultured in reactors/fermentors containing growth medium at 35° C.-38° C. for 10-18 hrs. The process is considered to be completed when the cell concentration is 4-5 billion/ml and the spore to vegetative cell ratio is 1:1. Upon completion of the incubation, the separately grown cultures are combined in the 1:1 ratio, a sucrose-gelatin protective medium is added, and the liquid is poured into flasks. The obtained preparation contains biomass of the B. subtilis VKPM B-8611 strain in the 5·10⁹ CFU/ml titer and biomass of the B. licheniformis VKPM B-8610 strain in the 5·10⁹ CFU/ml titer. 2.5-4.5 mg/ml of each of the filtrates of the aforementioned strains and 5.5-7.5 mg of the Lactobacillus fermentum 90 TS-4 (21) lectin-binding substance are added to the obtained bacterial composition.

### EXAMPLE 2

### Preparing the Preparation as a Lyophylisate

Laboratory technology: the strains are cultured on solid agar media in flasks or glass bottles in a thermostat-controlled shaker at 22° C. to 38° C. from 12-16 hrs. to up to 7 days. Upon completion of the incubation, the biomass grown on top of the growth medium is washed of with a protective medium containing a 10% glycerol solution. 2.5-4.5 mg/ml of each of the filtrates of the aforementioned strains and 5.5-7.5 mg of the Lactobacillus fermentum 90 TS-4 (21) lectin-binding substance are added to the obtained bacterial composition.

The liquid is then poured into vials (flasks) or into stainless steel cassettes and freeze-dried and dehydrated in a vacuum freeze-dryer or spray-dried.

Industrial technology: the strains are cultured in reactors/fermentors containing growth medium at 35° C.-38° C. for 10-18 hrs. The process is considered to be completed when the cell concentration is 4-5 billion/ml and the spore to vegetative cell ratio is 1:1. Upon completion of the incubation, the separately grown cultures are combined in a 2:1 ratio, a stabilizer is added, and 2.5-4.5 mg/ml of each of the filtrates of the aforementioned strains and 5.5-7.5 mg of the Lactobacillus fermentum 90 TS-4 (21) lectin-binding substance are added to the obtained bacterial composition.

The liquid is then poured into vials (flasks) or into stainless steel cassettes and freeze-dried and dehydrated in a vacuum freeze-dryer or spray-dried.

### EXAMPLE 3

### Preparing the Preparation in the Tablet Form

2.5-4.5 mg/ml of each of the filtrates of the B. subtilis VKPM B-8611 and B. licheniformis VKPM B-8610 strains and 5.5-7.5 mg of the Lactobacillus fermentum 90 TS-4 (21) lectin-binding substance are added to the bacterial composition resulting from the addition of the suspension medium components to the cultured B. subtilis VKPM B-8611 and B. licheniformis VKPM B-8610 strains, dehydrated in a vacuum freeze-dryer or a spray-dryer, combined with sugar granules and glidants (starch or calcium stearate), and pressed on rotary presses.

### EXAMPLE 4

### Preparing the Preparation in the Suppository Form

2.5-4.5 mg/ml of each of the filtrates of the B. subtilis VKPM B-8611 and B. licheniformis VKPM B-8610 strains and 5.5-7.5 mg of the Lactobacillus fermentum 90 TS-4 (21) lectin-binding substance are added to the bacterial composition resulting from the addition of the suspension medium components to the cultured B. subtilis VKPM B-8611 and B. licheniformis VKPM B-8610 strains, dehydrated in a vacuum freeze-dryer or a spray-dryer, combined with excipients (confectionary fat, paraffin, etc.) and molded in a special suppository-making machine.

### EXAMPLE 5

All variants and forms of the Balis biological preparation obtained in examples 1, 2, 3, and 4 are tested for safety on laboratory animals, for specific antagonistic activity against test-cultures—representatives of various groups of pathogenic and opportunistic microorganisms, and for resistance to antibiotics.

Balis biological preparation is safe. A safety study was conducted on 2 groups of mice: the first group was used to study Balis preparation; the second group was examined for safety of live culture filtrates. 10 mice weighing 15-16 g each were used in each version of the test and in control. To establish the safety of the Balis preparation, the content of the flask was dissolved in 0.5 ml of physiological solution and this dose was then administered orally to the mice in the first group. The preparation is considered to be safe if all the mice survive during 5 days of observation, and none of them develops the disease. The mice in the second group were administered 0.3 ml of sterile filtrates of Balis preparation by intraperetoneal injection weekly, over a 4-week period. In the beginning and at the end of the experiment, a drop of blood was drawn from the caudial veins of the mice to study the leukogram by obtaining the white blood cell count in the smears using the Romanowsky-Giemsa staining technique. The mice were weighed at the end of the experiment and their final weight was compared to their initial weight. A pathology study of the animals was also conducted. The mice in the first group survived through the first 5 days of the study, and no one became sick. The mice in the second group were alive at 30 days and gained 2.0-2.5 g. The pathology study showed an insignificant increase in the size of the spleen and no visible changes in other organs. The morphological study of the white blood cells revealed the following results: the leukograms of the mice in both first and second groups (blood drawn on the last day of the experiment) were practically identical to the initial leukograms.

Balis biological preparation exhibits a wide range of antagonistic activity against test-strains of pathogenic and opportunistic microorganism cultures. The study is conducted by the deferred antagonism method. The contents of the flask are dissolved in 1 ml of physiological solution. The resulting suspension is streak plated along the diameters of Petri dishes filled with Gauze #2 agar medium. The plate is incubated in a thermostat at 37° C. for 72 hrs. Then, test-microorganisms (500-million suspensions of the 24 hr.—culture in physiological solution) are streak plated on the culture grown in the Petri dish. The results are evaluated by the size of the areas where test-cultures have not grown 18 hrs. post incubation at 37° C.

Test-cultures simultaneously plated in Petri dishes with Gauze #2 agar medium and no culture of the present invention serve as control.

The optimum number of live cells per dose of the preparation is 1-5×10⁹ A further increase of the microbial cell amount does not significantly affect the antagonistic activity of the preparation against test-cultures of the microorganisms (see Table 3).

The lectin-binding substance modulates the adhesive activity of the yeast-like C. albicans type fungal test-cultures and opportunistic strains of the E. Coli type microorganisms towards vaginal epithelial cells of clinically healthy women. This statement can be illustrated with the following results (see Table 4).

Spore probiotics exhibit a set of properties that enable them to compete with pathogenic and opportunistic microorganisms. These properties include antagonistic activity, ability to adhere to the epithelial cells, a certain level of resistance to hydrochloric acid, bile, etc. However, the effect of these preparations on the tuberculosis pathogens has not yet been studied. In recent years, increased morbidity and mortality from tuberculosis coincided with elevated multiple drug resistance (MDR-TB) and the occurrence of mycobacteria tuberculosis strains with extensive resistance to drugs (XDR-TB). The clinical picture of drug-resistant tuberculosis develops in patients when the population of multiple drug resistant mycobacteria considerably surpasses the population of bacilli sensitive to antituberculosis drugs. Academia (M. tuberculosis), Vallee (M. bovis), Ravenal (M. bovis), and RSICS (Russian State Institute of Control and Standardization) (M. avium), reference strains obtained from L. A. Tarasevich RSICS were used to study the antagonistic activity of Balis preparation. M. bovis (#3. Ryazan) bovine strains that we isolated and identified by cultural, biochemical, and chemotaxonomic methods were also used as indicator strains (see Tables 5, 6). In addition, 20 drug-resistant M. tuberculosis strains were isolated and identified from 20 tuberculosis patients (Table 7). The study method of antagonistic activity of probiotic strains against mycobacteria tuberculosis involved the use of culture fluid filtrates obtained with sterilizing filters (see A. L. Lazovskaya et al., The effect of probiotics on pathogenic mycobacteria. The problems of tuberculosis and lung diseases. Published by “Medicina”, M, 2006, —#7. P.p. 25-27.) The results were evaluated during 25-30 days of incubation at 37° C. using Growth Inhibition Index (GII) of mycobacteria. GII was calculated as a ratio of colonies formed on the control medium with no added probiotic to the number of colonies formed in the medium with the probiotic. The maximum GII is considered to be 10.

The effect of spore probiotics on the mycobacterial drug sensitivity was studied on 20 clinical strains obtained from tuberculosis patients applying the aforementioned methods of treating spore probiotics. Drug sensitivity was determined pursuant to Order #109 of Sep. 21, 2003 “On Improvement of Antituberculous Measures in the Russian Federation.” The results are shown on the figure. The following pharmaceutical preparations in varying concentrations were used in the experiment: 1,2-streptomycin 10 and 25 μg/ml respectively; 3,4-isoniazid 1 and 10 μg/ml, 5-kanamycin 30 μg/ml; 6-ethambutol 2 μg/ml; 7-rifampicin 40 μg/ml; 8-ethionamide 30 μg/ml; 9-ofloxacin 2 μg/ml; 10-capreomycin 30 μg/ml; 11-PASA (para-aminosalicylic acid) 1 μg/ml.

The probiotics of this invention exhibited antagonistic activity against all the reference strains. As shown in Tables 5-7, the spore probiotics inhibited the growth of M. tuberculosis. Growth Inhibition Index (GII) of 20 mycobacteria tuberculosis strains fluctuated between 1 and 10.

A study of drug-sensitive isolates grown on media with sterile filtrates revealed that sensitivity to drugs was restored in 80% of cases, when 95% of initial cultures were resistant to 1, 2, 3, 4, 5, 6 and even 7 antituberculosis drugs. 20% of strains were resistant to 7-8 antibiotics, i.e. they were XDR-TB and MDR-TB. Out of 20 drug-resistant clinical strains of mycobacteria, sensitivity to one, two, three, four, or five pharmaceutical preparations was restored in 16 (80%) cases.

TABLE 1
Cultural, Morphological, and Biochemical Properties

| | B. subtilis | B. licheniformis |
|---|---|---|
| Properties | VKPM B-8611 | VKPM B -8610 |
| Growth in anaerobic | − | + |
| conditions | | |
| Fermentation glucose | + | + |
| arabinose | + | + |
| xylose | + | − |
| mannitol | + | + |
| Citrate utilization | + | + |
| Propionate utilization | − | + |
| Starch hydrolysis | + | + |
| Urea hydrolysis | − | − |
| Nitrate reduction | + | + |
| Formation of gas from NO₃⁻ in | − | + |
| anaerobic conditions | | |
| Methylene blue, decolorization | + | + |
| Arginine dihydrolase | − | + |
| Lecithinase | − | − |
| Hyaluronidase | − | − |
| Hemolytic activity | − | − |
| Formation of poly-β-oxybutyric | − | − |
| acid globules on glucose agar | | |

TABLE 2
Sensitivity to Antibiotics of the Bacillus Type Strains

| | Diameter of the culture's inhibited growth area, mm | |
|---|---|---|
| Preparation | B. subtilis | B. licheniformis |
| under study | VKPM B-8611 | VKPM B-8610 |
| PENICILLINS | | |
| Azlocillin | 19 ± 0.1 | 16 ± 0.1 |
| Amoxicillin | 19 ± 0.2 | 16 ± 0.1 |
| Ampicillin | 11 ± 0.2 | ∘ |
| Carbenicillin | 23 ± 0.5 | 20 ± 0.1 |
| Mezlocillin | 21 ± 0.2 | 15 ± 0.2 |
| Methicillin | 20 ± 0.1 | 10 ± 0.1 |
| Oxacillin | 15 ± 0.3 | 11 ± 0.1 |
| Benzylpenicillin | 7 ± 0.1 | 3 ± 0.2 |
| Piperacillin | 15 ± 0.3 | 11 ± 0.1 |
| Ticarcillin | 25 ± 0.4 | 21 ± 0.2 |
| CARBAPANEMS | | |
| Imipenem | 38 ± 0.2 | 30 ± 0.1 |
| CEPHALO- | | |
| SPORINS | | |
| Moxalactam | 11 ± 0.2 | 12 ± 0.3 |
| Cephalothin | 35 ± 0.1 | 21 ± 0.3 |
| Cefazolin | 25 ± 0.2 | 19 ± 0.2 |
| Cefamandole | 35 ± 0.3 | 15 ± 0.1 |
| Cefoxitin | 18 ± 0.1 | 13 ± 0.1 |
| Cefoperazone | 15 ± 0.2 | 13 ± 0.1 |
| Cefotaxime | 15 ± 0.1 | 9 ± 0.1 |
| Ceftazidime | 6 ± 0.3 | 13 ± 0.2 |
| Ceftizoxime | 3 ± 0.1 | 11 ± 0.1 |
| Ceftriaxone | 20 ± 0.4 | 12 ± 0.2 |
| Cefuroxime | 2 ± 0.1 | 3 ± 0.1 |
| AMINO- | | |
| GLYCOSIDES | | |
| Amikacin | 22 ± 0.3 | 17 ± 0.2 |
| Gentamicin | 25 ± 0.3 | 19 ± 0.2 |
| Kanamycin | 22 ± 0.2 | 21 ± 0.1 |
| Tobramycin | 25 ± 0.1 | 20 ± 0.2 |
| OTHER | | |
| Vancomycin | 12 ± 0.1 | 10 ± 0.1 |
| Clindamycin | 9 ± 0.2 | 12 ± 0.1 |
| Tetracycline | 26 ± 0.1 | 24 ± 0.1 |
| Chloramphenicol | 18 ± 0.3 | 10 ± 0.1 |
| Polymyxin B | 11 ± 0.1 | 10 ± 0.2 |
| Nitrofurantoin | 15 ± 0.2 | 20 ± 0.1 |
| Trimethoprim | 23 ± 0.1 | 25 ± 0.2 |
| Bactrim | 29 ± 0.2 | 31 ± 0.3 |
| Norfloxacin | 24 ± 0.2 | 25 ± 0.2 |
| Nalidixic acid | 21 ± 0.1 | 15 ± 0.1 |

TABLE 3
Antagonistic Activity of the Bacillus subtilis VKPM B-8611 and Bacillus licheniformis
VKPM B-8610 strains Against Pathogenic and Opportunistic Microorganisms

| | Variants of the biological preparation (ratio between | | | | |
|---|---|---|---|---|---|
| | Bacillus subtilis VKPM B-8611 and Bacillus | | | | |
| | licheniformis VKPM B-8610) × 10^{8×6} CFU/ml | | | | |
| Test culture | Areas of inhibited growth of test-strains (mm) | | | | |
| Source | 1:1 | 2:1 | 5:1 | 1:10 | 10:1 |
| Shigella Sonnei (n = 25) | 10-12 | 12-15 | 12-15 | 12-15 | 12-15 |
| (from dysentery patients) | | | | | |
| Salmonella typhimurium (n = 25) | 10-13 | 12-18 | 12-18 | 12-18 | 12-18 |
| (from salmonellosis patients) | | | | | |
| Escherichia coli 0:157:H7 (n = 13) | 9-10 | 11-13 | 11-13 | 11-13 | 11-13 |
| (from enterohemorrhagic colitis | | | | | |
| patients and from animals) | | | | | |
| Staphylococcus aureus (n = 20) | 10-15 | 15-20 | 15-20 | 15-20 | 18-20 |
| (from intestinal | | | | | |
| dysbacteriosis patients) | | | | | |
| Staphylococcus aureus (n = 45) | 12-16 | 15-18 | 15-20 | 18-20 | 22-25 |
| (pyoinflammatory diseases) | | | | | |
| Staphylococcus aureus (n = 29) | 15-18 | 15-20 | 18-22 | 18-22 | 20-25 |
| (vaginal dysbiosis) | | | | | |
| Proteus vulgaris (n = 18) | 10-15 | 15-20 | 15-20 | 15-20 | 15-20 |
| (intestinal dysbacteriosis) | | | | | |
| Proteus vulgaris (n = 18) | 10-12 | 12-15 | 12-15 | 12-15 | 12-15 |
| (pyelonephritis patients) | | | | | |
| Proteus mirabilis (n = 15) | 15-18 | 18-20 | 18-20 | 18-20 | 18-20 |
| Candida albicans (n = 42) | 25-30 | 25-30 | 25-30 | 25-30 | 25-30 |
| (intestinal dysbacteriosis) | | | | | |
| Candida albicans (n = 20) | 25-30 | 25-30 | 25-30 | 25-30 | 25-30 |
| (vaginal dysbiosis) | | | | | |
| Escherichia coli (n = 18) | 20-22 | 22-25 | 22-25 | 22-25 | 22-25 |
| (pyelonephritis patients) | | | | | |
| Escherichia coli (n = 15) | 10-12 | 12-15 | 12-15 | 12-15 | 12-15 |
| (pyoinflammatory diseases) | | | | | |
| Streptococcus (n = 17) | 10-12 | 12-15 | 12-15 | 12-15 | 12-15 |
| ((pyoinflammatory diseases) | | | | | |

TABLE 4
Modulating Effect of Culture Fluid Concentrate Fractions of L. fermentum, Strain
90 TS-4 (21), Clone 3 on Test-Cultures During Adhesion to Vaginal Epithelium

| | Concentrate (PM-20) CF L. fermentum | | | |
|---|---|---|---|---|
| | 90-TS-4 (21) clone 3 (Cₚᵣₒₜₑᵢₙ = 150 μ/ml) | | | |
| | Fraction capable of | | Fraction incapable of | |
| | reacting with Con A | | reacting with Con A | |
| Type and strain of | M ± m | | M ± m | |
| microorganism | control | experiment | control | experiment |
| C. albicans | 4.33 ± 0.4 | 1.2 ± 0.2* | 1.26 ± 0.2 | 0.64 ± 0.12* |
| 04-703 [symbol] | | | | |
| (vaginal isolate) | | | | |
| C. albicans | 13.04 ± 1.8 | 8.62 ± 1.12* | 2.74 ± 0.5 | 4.76 ± 1.02 |
| (oral isolate) | | | | |
| E. coli K-12 | 12.35 ± 0.84 | 12.12 ± 1.34 | 12.35 ± 0.84 | 20.94 ± 1.51 |
| E. coli 89-1449 | 33.92 ± 2.2 | 19.92 ± 1.42* | 13.76 ± 1.6 | 23.64 ± 2.9 |
| (intestinal isolate) | | | | |

*significant reduction in adhesion of test-cultures to vaginal epithelium

TABLE 5
Antagonism of Balis Preparation and its Components (Autoclave Culture
Suspension) towards Reference Strains and M. bovis Strain

| | | IBR | | |
|---|---|---|---|---|
| #p/p | Type of mycobacteria | B. lichenifor. | B. subtilis | Balis |
| 1 | M. tuberculosis (Academia) | 10 | 10 | 10 |
| 2 | M. avium (RSICS) | 5 | 3 | 3 |
| 3 | M. bovis (Vallee) | 0 | 0 | 0 |
| 4 | M. bovis (Ravenal) | 3 | 3 | 4 |
| 5 | M. bovis (#3 Ryazan) | 1 | 1 | 1.5 |

TABLE 6
Antagonism of BALIS Preparation and its Components
(Culture Suspension Filtrate) towards Reference
Strains and Bovine M. bovis Strain

| | | IBR | | |
|---|---|---|---|---|
| #p/p | Type of mycobacteria | B. lichenifor. | B. subtilis | Balis |
| 1 | M. tuberculosis (Academia) | 10 | 10 | 10 |
| 2 | M. avium (RSICS) | 10 | 10 | 10 |
| 3 | M. bovis (Vallee) | 0 | 0 | 0 |
| 4 | M. bovis (Ravenal) | 10 | 10 | 10 |
| 5 | M. bovis (#3 Ryazan) | 5 | 8 | 8 |

TABLE 7
Antagonism of BALIS Preparation and its Components B. subtilis
and B. licheniformis (Culture Suspension Filtrate) Towards Clinical
Mycobacterium tuberculosis Strains from Infected People

IBR

| # clinical | B. subtilis VKPM B-8611and | |
|---|---|---|
| strain | B. licheniformis VKPM B-8610 | Balis |
| 1 | 6 | 6 |
| 2 | 5 | 5 |
| 3 | 9 | 9 |
| 4 | 10 | 10 |
| 5 | 10 | 10 |
| 9 | 10 | 10 |
| 10 | 3 | 3 |

We claim:

1. A biological preparation for prevention and treatment of infectious diseases and dysbiosis of various etiologies, primarily tuberculosis and the concomitant candidiasis, comprising a live microbial mass of Bacillus subtilis VKPM B-8611 and Bacillus licheniformis VKPM B-8610 strains,
wherein said biological preparation further comprises live culture filtrates of said Bacillus subtilis and Bacillus licheniformis strains and a lectin-binding substance comprising a supernatant of live microbial mass culture fluid of the Lactobacillus fermentum 90 TS-4(21) strain in the following component ratio of:
the live microbial mass of the Bacillus subtilis VKPM B-8611 strain—at least 5×10⁸ CFU;
the live microbial mass of the Bacillus licheniformis VKPM B-8610 strain—at least 2×10⁸ CFU;
a lyophilized filtrate of the live microbial mass of the Bacillus subtilis VKPM B-8611 strain, 2.5-4.5 mg;
a lyophilized filtrate of the live microbial mass of the Bacillus licheniformis VKPM B-8610 strain, 2.5-4.5 mg; and
a lyophilized lectin-binding substance comprising a culture fluid supernatant of live microbial mass of a Lactobacillus fermentum 90 TS-4(21) strain with the molecular weight exceeding 20 kDa, 5.5-7.5 mg.

2. The biological preparation of claim 1, wherein infectious diseases comprise chronic tuberculosis, intestinal infections, nosocomial surgical infections, candidiasis, and dysbiosis.

3. The biological preparation of claim 2, wherein chronic tuberculosis is caused by XDR TB mycobacteria with extensive drug resistance.
